(19) United States
(12) Patent Application Publication
(10) Pub. No.: US 2026/0271867 A1
(43) Pub. Date: Sep. 17, 2026

(54) NEW SPORELESS PLEUROTUS STRAIN

(71) Applicant: Somycel, Langeais (FR)

(72) Inventors: Aniça AMINI, Saint-Pierre-Des-Corps (FR); Ségolène PÉGÉ, Joue-Les-Tours (FR); Mark WACH, Allison Park, PA (US)

(73) Assignee: Somycel, Langeais (FR)

(21) Appl. No.: 19/168,138

(22) PCT Filed: Mar. 28, 2024
(86) PCT No.: PCT/EP2024/058615

§ 371 Date: Sep. 23, 2025

(30) Foreign Application Priority Data
Mar. 28, 2023 (EP) 23305429.5

(51) Int. Cl. A01H 15/00; A01G 18/50; A01H 1/02

(52) U.S. Cl. CPC A01H 15/00; A01G 18/50; A01H 1/021

(57) ABSTRACT

The present invention relates to the development of a new sporeless Pleurotus strain designated FiBABH-334, a representative culture thereof having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number I-5939. This sporeless Pleurotus strain displays an excellent yield weight of the harvested crop and a very good shelf-life of the mushroom products. The present invention also relates to cultures obtained, descended, or otherwise derived from this strain. More particularly, the present invention relates to cultures comprising at least one haploid set of chromosomes of said strain, especially to monokaryons thereof or to F1 hybrids produced by mating this monokaryon with a second Pleurotus line. The invention further relates to methods of use of the cultures described hereinabove.

### FIELD OF THE INVENTION

The present invention relates to the development of a new sporeless Pleurotus strain designated FIBABH-334, a representative culture thereof having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number 1-5939. This sporeless Pleurotus strain displays an excellent yield weight of the harvested crop and a very good shelf-life of the mushroom products. The present invention also relates to cultures obtained, descended, or otherwise derived from this strain. More particularly, the present invention relates to cultures comprising at least one haploid set of chromosomes of said strain, especially to monokaryons thereof or to F1 hybrids produced by mating this monokaryon with a second Pleurotus line. The invention further relates to methods of use of the cultures described hereinabove.

### BACKGROUND OF THE INVENTION

Pleurotus species are commercially essential mushrooms and widely cultivated throughout the world. The production of Pleurotus mushrooms alone accounts for around 25% of total cultivated mushroom globally. In America and Europe, Pleurotus species are considered specialty mushrooms, whereas, in Korea, their cultivation is economically profitable, and it is one of the highly consumed species. Pleurotus mushrooms are rich in proteins, dietary fiber, essential amino acids, carbohydrates, water-soluble vitamins, and minerals. These mushrooms are abundant in functional bioactive molecules, though to influence health. Pleurotus mushrooms are finding unique applications as flavoring, aroma, and excellent preservation quality. Apart from its unique applications, Pleurotus mushrooms have a unique status delicacy with high nutritional and medicinal values. Finally, Pleurotus species can be grown easily due to their fast colonization nature on diversified agro-substrates and their biological efficiency 100% (Raman et al, 2021).

One of the main problems in growing Pleurotus strains is the occurrence of respiratory problems among the people who are harvesting and handling them. The symptoms of this disease are identical to the “mushroom workers lung», a more general term that is also used to identify a number of other respiratory diseases is extrinsic allergic alveolitis. Several authors have reported that the occurrence of this disease is associated with exposure of people to spores of Pleurotus species (Baars J. J. P. et al, 2000).

Spore production by Pleurotus strains is immense. Traditional and present strains produce between 200 and 660 million spores per gramme of tissue per 24 hrs. Spore production by “poorly sporulating” or “low sporulating” strains is ten times lower (Sonnenberg et al, 1996) but still high enough to yield spore concentrations that are well above the threshold values for the induction of symptoms of extrinsic allergic alveolitis (Baars J. J. P. et al, 2000).

In this context, sporeless Pleurotus strains are needed to improve the health of the mushroom workers.

Several sporeless Pleurotus mushrooms have already been produced:
The Somycel 3200 and 3210 strains (US plant patent U.S. Pat. No. 4,658,083) described in the 1980's were sporeless Pleurotus strains having a typical decurrent lamellae over the major part of the height of the foot, and more developed lamellae than other prior art strains. Yet, these strains have a cornet, trumpet or curled shape that impair their easy packaging. Moreover, they have a very long stipe (U.S. Pat. No. 4,658,083).
The most famous sporeless Pleurotus strain is the so-called “Spoppo” strain described notably in the plant patent application US2006/0010549. It has sporeless basidia like Somycel 3210, but has shell-shaped, well-developed fleshy mushrooms with heavy caps, unlike Somycel 3210. Its shell-formed caps are large (until 15 cm in diameter), usually curved and attached eccentrically to the stipe. The length of its stipes is quite high (54.8 mm on average, US2006/0010549) and the total height of the mushrooms is of about 64.6 mm (second flush, present results). Its yield is very good.
More recently, the so-called “Allerpo” sporeless Pleurotus strain was described (US Plant Patent pp 32864). Its morphology is similar to Spoppo mushrooms, as the two strains originate from the same breeding program. In particular, their caps also have a shell-shaped morphology and are also usually curved. Allerpo's cap color is however darker and browner than Spoppo's cap color. The length of their stipes is quite high (51.5 mm on average, pp 32864) and the total height of the mushrooms is of about 65.2 mm (first flush, present results). Its yield is the same as Spoppo's strain yield.

These three sporeless Pleurotus strains of the prior art are therefore morphologically characterized by a curved cap and a long stipe, which triggers a total height superior to 50 mm, a great part of it being the stipe. The tall height of their stipes and the curved-shape of their caps affect packaging of the mushrooms, which are very perishable products that should be processed and carried adequately and cautiously to preserve their quality, extend their shelf-life and ensure satisfactory commercialization.

In this context, there was therefore a need to provide sporeless Pleurotus strains that have a flatter cap and a smaller stipe, so that the final mushroom products can be more easily stored and transported in big quantities (taking less space), and so that the edible part of the mushroom is optimized to ensure less waste (i.e., more valuable product) once the stipe part is cut out.

The present invention solves this need, by providing a new sporeless Pleurotus strain whose mushrooms have a cap flatter than Spoppo's and Allerpo's mushroom caps, and a very short stipe. These two interesting traits advantageously facilitate packaging of the mushrooms and their transportation (more quantity can be packaged in the same volume without affecting the quality of the mushroom edible parts) and also allow to reduce the rate of waste (see examples below). Importantly, this new strain has the same production yield as Spoppo's and Allerpo's (see examples below), that are known to be excellent.

### DETAILED DESCRIPTION OF THE INVENTION

### Pleurotus Mushroom Strain of the Invention

The present invention proposes a new Pleurotus ostreatus strain that does not release any spores, and that has a great economic value, because of its advantageous high yield, flat cap and short stem.

This strain has been called “FiBABH-334”. A representative culture thereof has been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023.

This strain has been obtained by back-crossing the TPLO4 strain (PVR 20173194) with the ATTC-58937 mutant strain. TPLO4 was partly restored by doing two successive back-crosses.

Mushrooms produced in crops by strain FiBABH-334 are about 23% by weight (S. D. ±2.45) (calculated as the number of kg mushrooms/100 kg of substrate) over 2 flushes. They have a short size (27.5 mm for the first flush, 36.4 mm for the second flush, that is about half the size of Spoppo's mushrooms, see the examples below).

Cap color measurements on mushrooms of FiBABH-334 produced L-a-b color of L: 69.3 (S. D±4.1) a:1.9 (S.D. ±0.8) b:14.1 (S.D. ±1.8) when measurements were taken on 30 mushrooms using a Minolta Chromameter.

As described in the experimental part below, the FiBABH-334 strain has an excellent yield (23.26% total), as the leading commercial strain, Spoppo. The FiBABH-334 strain also have a significantly reduced rate of waste (ratio of cut/uncut weight of the mushrooms) and therefore a better economic value than Spoppo's.

The Pleurotus strain of the present invention can be considered as sporeless as it produces no visible spores as observed on a spore print during 24 hours. In fact, it has a spore production of at least 60.106 time less than the parent TPLO4 strain. The Pleurotus strain of the invention is able nevertheless to produce 20-50 spores during 24 h on spore print, so that they can be germinated.

In a first aspect, the present invention targets a sporeless Pleurotus strain comprising at least one haploid set of chromosomes of the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023. The deposit of a culture of the Pleurotus strain FiBABH-334, as disclosed herein, has been made by Somycel, 4 Rue Carnot-ZI Sud, 37130 Langeais, with the Collection Nationale de Cultures de Microorganismes (CNCM).

The culture deposited was taken from the same culture maintained by Somycel, Langeais, France, the assignee, since prior to the filing date of this application, and the inventors and assignee have received authorization to refer to this deposited biological material in any and all patent applications. All restrictions upon the deposit have been removed, and the deposit is intended to meet all deposit requirements of the Budapest Treaty. The date of deposit was Mar. 21, 2023. Moreover, the deposit will be maintained in the depository for a period of 30 years, or 5 years after the last request, or for the effective life of any patent, whichever is longer, and will be replaced as necessary during this period. The culture of this deposit will be irrevocably and without restriction of condition released to the public upon the filing of the patent application or upon the issuance of a patent, whichever is required by the applicable patent laws.

Mushroom cultures are most reliably identified by their genotypes, in part because successful cultivar strains are required by the market to conform to a narrow phenotypic range. The genotype can be characterized through a genetic marker profile, which can identify isolates (clones or subcultures) of the same line, strain or culture, or a genealogically related culture including a descendent or a culture derived entirely from an initial culture, or additionally can be used to determine or validate a strain development pedigree over generations.

In Inventor's experience in evaluating whole genome sequences of many dozens of diverse Pleurotus lines and strains, a typical number of SNP markers distinguishing any two unrelated monokaryons is roughly 300,000. This means that transmission of even 1% of a set of chromosomes or genotypic markers in a genealogy still represents about 3,000 distinctive identifying markers for a relationship to a particular monokaryon. 200-300 markers, can establish identity, paternity, and derivation among cultures beyond question, while many thousands of available SNP markers can cumulatively provide a robustly-supported method for establishing genealogical relatedness over multiple generations.

Means of obtaining genetic marker profiles using diverse techniques including whole genome sequencing (WGS) plus Single Nucleotide Polymorphism (SNP) marking and Sequence Characterized Amplified Region (SCAR) marking are well known in the art. Since both approaches can analyse the sequences of specific loci, both provide identical results for any locus (note that in dikaryon analysis, WGS provides more insight into the distribution of SNPs on the haploid sequences; i.e., confirmation of allelic sequences).

The whole genomic sequence of the strain FiBABH-334 has been obtained and, consequently, about 95% (about 34.4 to 35.6 Mb) of the entire DNA sequence genotype of the strain FiBABH-334 is known to the Assignee with certainty.

A brief excerpt of the genotype of the strain FiBABH-334 at numerous sequence-characterized marker loci distributed at intervals along each of the 11 chromosomes of FiBABH-334 is provided in Table A. Only for information, the sequences of the same marker loci are provided for the PC15 strain, for the Spoppo strain, and for the Allerpo strain. This table shows that the strain of the invention differs from the Spoppo strain by at least 298 SNPs markers, from the Allerpo strain by at least 298 SNPs markers.

TABLE A

| Scaffold ID | Ref Pos | PC15 | SPOPPO | ALLERPO | FiBABH-334 |
|---|---|---|---|---|---|
| scaffold_1 | 96 221 | TGGATGCGG | TGGATGCGS | TGGATGCGS | TGGAYGCGS |
| scaffold_1 | 199 734 | ACTGTAATT | ACTGTAATT | ACTGTAATT | ACTGYARTT |
| scaffold_1 | 306 572 | CCCCAACGA | CCCCRACGA | CCCCRACGA | CCCCAACGA |
| scaffold_1 | 537 787 | CCAATGCGC | CCAAYGCGC | CCAAYGCGC | CCAATGCGC |
| scaffold_1 | 697 612 | CACATTGCC | CACAWTGCC | CACAWTGCC | CACATTGCC |
| scaffold_1 | 849 984 | TATCATTAA | TATCGTTGA | TATCGTTGA | TATCRTTRA |
| scaffold_1 | 1 000 571 | TGAAGCCGA | TGAAACCAA | TGAAACCAA | TGAARCCRA |
| scaffold_1 | 1 001 095 | TTATGGTCA | TTATAGTTC | TTATAGTTC | TTATRGTTM |
| scaffold_1 | 1 104 031 | TGGTTACTT | TGGT----- | TGGT----- | TGGTtactt |
| scaffold_1 | 1 199 962 | TGACAACGC | TGACRACGC | TGACRACGC | TGACAACGC |
| scaffold_1 | 1 300 534 | TTCAGAACA | TTCAAAACA | TTCAAAACA | TTCARAACA |
| scaffold_1 | 1 500 272 | GGTAGCTGT | GGTAGCTGT | GGTAGCTGT | GGTARCTGT |
| scaffold_1 | 1 910 774 | CCGAGAACA | CCGAGAACA | CCGAGAACA | CCGAKAACA |
| scaffold_1 | 2 138 358 | ATTCGAGTA | ATTCRAGTA | ATTCRAGTA | ATTCGAGTA |
| scaffold_1 | 2 189 053 | CTACTATGA | CTACTATGA | CTACTATGA | CTACYATGA |
| scaffold_1 | 2 351 257 | AGAT-AAAC | AGATAAAAC | AGATAAAAC | AGATaAAAC |
| scaffold_1 | 2 431 023 | RAGTCATTG | RAGTCATTG | RAGTCATTG | RAGTMATTG |
| scaffold_1 | 2 489 664 | CAAGTCCCT | CAAGTCCCT | CAAGTCCCT | CAAGYCCCT |
| scaffold_1 | 2 543 688 | TTTGTCAAC | TTTGTCAAC | TTTGTCAAC | TTTGWCAAC |
| scaffold_1 | 2 750 040 | CCGACCCCC | CCGAYCYCC | CCGAYCYCC | CCGACCYCC |
| scaffold_1 | 2 850 298 | TAATGCAGA | TAATGCAGA | TAATGCAGA | TAATSCAGA |
| scaffold_1 | 3 002 856 | AAGTATCTC | ARGTMTCTC | ARGTMTCTC | ARGTATCTC |
| scaffold_1 | 3 002 982 | GGGAGCTCT | GGGAGCTCT | GGGAGCTCT | GGGAACTCT |
| scaffold_1 | 3 003 067 | TGCCGAAAA | YGCCGAAAA | YGCCGAAAA | YGCCRAAAA |
| scaffold_1 | 3 100 582 | CTCACGAGA | CTCAYGAGA | CTCAYGAGA | CTCACGAGA |
| scaffold_1 | 3 250 614 | TGTACGTAT | TGTACGTAT | TGTACGTAT | TGTAYGTAT |
| scaffold_1 | 3 554 828 | ATCCACAGA | ATCCRCAGA | ATCCRCAGA | ATCCACAGA |
| scaffold_1 | 3 718 862 | CCACGCCCG | CCACGCCCG | CCACGCCCG | CCACACCCG |
| scaffold_1 | 4 016 112 | AGTACCTAA | AGTACCTAA | AGTACCTAA | AGTRCYTAA |
| scaffold_1 | 4 103 946 | TTCACGGCC | TTCACGGCC | TTCACGGCC | TTCAYGGCC |
| scaffold_1 | 4 200 012 | ″---------″ | ″---------″ | ″---------″ | aggtatcta |
| scaffold_1 | 4 299 883 | CTGTGCTCT | CTGTRCTCT | CTGTRCTCT | CTGTGCTCT |
| scaffold_1 | 4 403 549 | AAGTAGCGA | AAGTRGCGA | AAGTRGCGA | AAGTAGCGA |
| scaffold_1 | 4 503 548 | GCACGACCG | GCACGACCG | GCACGACCG | GCACSACCG |
| scaffold_1 | 4 602 050 | GAGCGAGTT | GAGCGAGTT | GAGCGAGTT | GAGCRAGTT |
| scaffold_1 | 4 700 048 | TCAGTCTTC | TCAGYCCTC | TCAGYCCTC | TCAGTCCTC |
| scaffold_1 | 4 800 425 | TCTTACCTT | TCTTRCMTT | TCTTRCMTT | TCTTACCTT |
| scaffold_2 | 10 240 | CGTCAAATA | CGTCRAATA | CGTCRAATA | CGTCAAATA |
| scaffold_2 | 35 109 | GATTCCTCT | GATTCCTCT | GATTCCTCT | GATTYCTCT |
| scaffold_2 | 106 133 | GTTGGCGGC | GTTGGCGGC | GTTGGCGGC | GTTGRCRGC |
| scaffold_2 | 115 244 | CCCATATTG | CCCATACCG | CCCATACCG | CCCAYACCG |
| scaffold_2 | 200 064 | CAGTCCACG | YAGTYCACG | YAGTYCACG | YAGTCCACG |
| scaffold_2 | 300 768 | ″---------″ | gtgcgaatc | gtgcgaatc | ″---------″ |
| scaffold_2 | 301 042 | CCTGTTTTG | CCTGCTTTG | CCTGCTTTG | CCTGTTTTG |
| scaffold_2 | 351 393 | TCAGCGCCT | TCAGCGCCT | TCAGCGCCT | TCAGYGCCT |
| scaffold_2 | 400 472 | AGTC-TTTT | AGTCtTTTT | AGTCtTTTT | AGTC-TTTT |
| scaffold_2 | 400 694 | CCTGCGCAA | CCTGGGCAA | CCTGGGCAA | CCTGCGCAA |
| scaffold_2 | 500 471 | GCTGCGAGC | GCTGCGAGC | GCTGCGAGC | GCTGYGAGC |
| scaffold_2 | 600 093 | TGACGCGAA | TGACRCGAA | TGACRCGAA | TGACGCGAA |
| scaffold_2 | 701 546 | GCGAGGTAG | GCGAGGTAG | GCGAGGTAG | GCGASGTAG |
| scaffold_2 | 810 117 | ATGACGACG | AYGAYGACG | AYGAYGACG | AYGACGACG |
| scaffold_2 | 950 394 | CCATAATCT | CCATAATCT | CCATAATCT | CCATRATCT |
| scaffold_2 | 1 050 040 | CCGCCCATG | CCGCSCATG | CCGCSCATG | CCGCCCATG |
| scaffold_2 | 1 101 245 | GGGAGCATG | GGGAACATG | GGGAACATG | GGGAGCATG |
| scaffold_2 | 1 257 662 | CTCCGAGCT | CTCCGAGCT | CTCCGAGCT | CTCCRRGCT |
| scaffold_2 | 1 401 558 | TATATTGCC | TATATTGCC | TATATTGCC | TATAYTGCC |
| scaffold_2 | 1 502 395 | CGGCGAGGG | CGGCGAGGG | CGGCGAGGG | CGGCRAGGG |
| scaffold_2 | 1 607 156 | TTCATGTAC | TTCATGTAC | TTCATGTAC | TTCAYSTAC |
| scaffold_2 | 1 748 426 | CCATCGTTC | CCATYGTTC | CCATYGTTC | CCATCGTTC |
| scaffold_2 | 1 853 370 | ACCCGCGCC | ACCCGCGCC | ACCCGCGCC | ACCCRCGCC |
| scaffold_2 | 1 958 640 | ACCCCAAAC | ACCCCAAAC | ACCCCAAAC | ACCCYAAAC |
| scaffold_2 | 2 081 744 | GGGCGTACG | GGGCGTACG | GGGCGTACG | GGGCRTACG |
| scaffold_2 | 2 194 676 | CATCACGCG | CATCACGCG | CATCACGCG | CATCMCGCG |
| scaffold_2 | 2 302 065 | GTCTCGGTG | GTCTCGGTG | GTCTCGGTG | GTCTYGGTG |
| scaffold_2 | 2 384 771 | AGACCCGCC | AGACCCGCC | AGACCCGCC | AGACMCGCC |
| scaffold_4 | 1 439 | AAGTCGCTC | AAGTCGCTC | AAGTCGCTC | AAGTYGCTC |
| scaffold_4 | 70 039 | TGTGCCGCC | TGTGCCGCT | TGTGCCGCC | TGTGYCGCY |
| scaffold_4 | 133 062 | AGATAAAGC | AGATAAAGC | AGATAAAGC | AGATRAAGC |
| scaffold_4 | 229 536 | CGGACAACC | CGGACAACC | CGGACAACC | CGGAYAACC |
| scaffold_4 | 300 004 | GTGCGGGTT | GTGCAGGTT | GTGCAGGTT | GTGCRGGTT |
| scaffold_4 | 400 029 | AGCCGGGAA | AGCCTGGAA | AGCCTGGAA | AGCCKGGAA |
| scaffold_4 | 450 080 | ATTCGGATA | ATTCAGATA | ATTCAGATA | ATTCRGATA |
| scaffold_4 | 500 644 | ATGAGCCAG | ATGAACCAG | ATGAACCAG | ATGARCCAG |
| scaffold_4 | 550 036 | AATCGAGGG | AATCAAGGG | AATCAAGGG | AATCRAGGG |
| scaffold_4 | 600 040 | TCACGGGAG | TCACAGGAG | TCACAGGAG | TCACRGGAG |
| scaffold_4 | 650 358 | CCCACCTAA | CCCAGCTAA | CCCAGCTAA | CCCAKCTAA |
| scaffold_4 | 700 028 | TCGGAACCA | TCGGGACCA | TCGGGACCA | TCGGRACCA |
| scaffold_4 | 751 391 | ATCTACGGA | ATCTCCGGA | ATCTCCGGA | ATCTMCGGA |
| scaffold_4 | 850 461 | GCCCATTGT | GCCC-TTGT | GCCC-TTGT | GCCCaTTGT |
| scaffold_4 | 851 262 | GTCAGAGTT | GTCAAAGTT | GTCAAAGTT | GTCARAGTT |
| scaffold_4 | 950 245 | CGAGAGACT | CGAGGGACT | CGAGGGACT | CGAGRGACT |
| scaffold_4 | 1 050 047 | CTAACATCA | CTAATATCA | CTAATATCA | CTAAWATCA |
| scaffold_4 | 1 150 722 | GGACGTGCC | GGACAKGCC | GGACAKGCC | GGACRKGCC |
| scaffold_4 | 1 250 087 | CTGCTGCCT | CTGCCGCCT | CTGCCGCCT | CTGCYGCCT |
| scaffold_4 | 1 355 126 | ATGAGTCGA | ATGATTCGA | ATGATTCGA | ATGAKTCGA |
| scaffold_4 | 1 500 621 | TCAATCTCG | TCAACCTCG | TCAACCTCG | TCAAYCTCG |
| scaffold_4 | 1 600 275 | TAGTCGCGT | TAGTCGCGT | TAGTCGCGT | TAGTYGCGT |
| scaffold_4 | 1 701 300 | TCCCCCGCG | TCCCYCGCG | TCCCYCGCG | TCCCCCGCG |
| scaffold_4 | 1 800 057 | TCTGCTATA | TCTGYTATA | TCTGYTATA | tctgttATA |
| scaffold_4 | 1 900 351 | TCTGCTGTG | TCTGCTGTG | TCTGCTGTG | TCTGYTGTG |
| scaffold_4 | 2 100 071 | GCTCCAACA | GCTCTAACA | GCTCTAACA | GCTCYAACR |
| scaffold_4 | 2 201 195 | ATCCGAACT | ATCCAAACT | ATCCAAACT | ATCCRAACT |
| scaffold_4 | 2 350 060 | GTTTTGGGG | GTTTYGGGG | GTTTYGGGG | GTTTTGGGG |
| scaffold_4 | 2 450 027 | GTCCGCTAG | GTCCRCTAG | GTCCRCTAG | GTCCGCTAG |
| scaffold_4 | 2 555 355 | CAATCGGCA | CAATYGGCA | CAATYGGCA | CAATTGGCA |
| scaffold_4 | 2 700 124 | GACGGCGGA | GACGCCAGA | GACGCCAGA | GACGSCRGA |
| scaffold_4 | 2 850 002 | GCCCAAGAC | GCCCRAGAC | GCCCRAGAC | GCCCAAGAC |
| scaffold_4 | 2 950 375 | TGTGCACAG | TGTGYACAG | TGTGYACAG | TGTGTACAG |
| scaffold_4 | 3 100 104 | GCACGGCAG | GCACRGCAG | GCACRGCAG | GCACAGCAG |
| scaffold_4 | 3 200 115 | CGGTGATGC | CGGTRATGC | CGGTRATGC | CGGTAATGC |
| scaffold_4 | 3 293 492 | GTCTGCATA | GTCTRCATA | GTCTRCATA | GTCTACATA |
| scaffold_4 | 3 301 886 | GCCGATTTC | GCCGRTTTC | GCCGRTTTC | GCCGATTTC |
| scaffold_4 | 3 405 248 | TCCACGGTC | TCCACGGTC | TCCACGGTC | TCCAYGGTC |
| scaffold_4 | 3 499 995 | AAAACCCCA | AAAAMCCCA | AAAAMCCCA | AAAACCCCA |
| scaffold_4 | 3 590 779 | TGTCAGGAC | TGTCSGGMC | TGTCSGGMC | TGTCRGGAC |
| scaffold_5 | 65 619 | ACCGATTTC | ACCGATTTC | ACCGATTTC | ACCGRTTTC |
| scaffold_5 | 102 801 | GGGGGAAAA | GGGGGAAAA | GGGGGAAAA | GGGGRAAAA |
| scaffold_5 | 210 025 | TATGGGTTT | TATGAGTTT | TATGAGTTT | TATGRGTTT |
| scaffold_5 | 315 122 | CCTCGTCAG | CCTCATCAG | CCTCATCAG | CCTCGTCAG |
| scaffold_5 | 415 064 | TGATCCCAC | TGATACTAC | TGATACTAC | TGATMCTAC |
| scaffold_5 | 501 136 | CAGTATCGA | CAGTATCGA | CAGTATCGA | CAGTRTCGA |
| scaffold_5 | 610 044 | TCCACGACT | TCCACGACT | TCCACGACT | TCCAYGACT |
| scaffold_5 | 720 036 | ATTCTGGCT | ATTCCGGCT | ATTCCGGCT | ATTCYGGCT |
| scaffold_5 | 850 049 | CTTTACATT | CTTTGCATT | CTTTGCATT | CTTTRCATT |
| scaffold_5 | 950 014 | AGACGCCGA | AGACRCCGA | AGACRCCGA | AGACGCCGA |
| scaffold_5 | 1 050 151 | GTTAGGTGG | GTTARGTGG | GTTARGTGG | GTTAGGTGG |
| scaffold_5 | 1 200 002 | TCATTTGCA | TCATYTGCA | TCATYTGCA | TCATTTGCA |
| scaffold_5 | 1 303 060 | GCAGAGAGG | GCAGWGAGG | GCAGWGAGG | GCAGAGAGG |
| scaffold_5 | 1 402 882 | TTCATCTTT | TTCATCTTT | TTCATCTTT | TTCAWYYTT |
| scaffold_5 | 1 500 000 | CTTTGTGGG | CTTTRTGGG | CTTTRTGGG | CTTTGTGGG |
| scaffold_5 | 1 600 062 | AAAGCTGAT | AAAGCTGAT | AAAGCTGAT | AAAGYTGAT |
| scaffold_5 | 1 701 004 | GTTCAGTAC | GTTCGGTAC | GTTCGGTAC | GTTCRGTAC |
| scaffold_5 | 1 801 090 | AGAAAGCCA | AGAAGGCCA | AGAAGGCCA | AGAARGCCA |
| scaffold_5 | 1 900 933 | TATGGCCAA | TRTGACCAA | TRTGACCAA | TATGRCCAA |
| scaffold_5 | 2 000 043 | CAACTATGT | CAACYATGY | CAACYATGY | CAACCATGT |
| scaffold_5 | 2 100 106 | AGCCACTAA | AGCCRCTAA | AGCCRCTAA | AGCCACTAA |
| scaffold_5 | 2 706 958 | CGCGCACAC | CGCGCACAC | CGCGCACAC | CGCGYACAC |
| scaffold_5 | 2 751 547 | CCTCACCAT | CCTCACCAT | CCTCACCAT | CCTCRCCAT |
| scaffold_5 | 2 942 325 | TGCTAGTCT | TGCTRGTCT | TGCTRGTCT | TGCTAGTCT |
| scaffold_5 | 3 042 032 | CCTAACGAC | CCTARCGAC | CCTARCGAC | CCTAGCGAC |
| scaffold_5 | 3 150 117 | GTCCGTGCA | GTCCRTGCA | GTCCRTGCA | GTCCGTGCA |
| scaffold_5 | 3 250 047 | CCGTAAGCA | CTGTRAGCA | CTGTRAGCA | CTGTAAGCA |
| scaffold_5 | 3 350 862 | GCGAATATA | GCGARTATA | GCGARTATA | GCGAGTATA |
| scaffold_5 | 3 540 680 | AGACACCAA | AGACACCAA | AGACACCAA | AGACWCCAA |
| scaffold_6 | 25 559 | TGTGTTATT | TGTGTTATT | TGTGTTATT | TGTGYTATT |
| scaffold_6 | 100 432 | AGCTTGTGC | AGCTYGTGC | AGCTYGTGC | AGCTCGTGC |
| scaffold_6 | 200 133 | CCAACGCTG | CCAACGCTG | CCAACGCTG | CCAAYGCTG |
| scaffold_6 | 300 240 | CCGGGTGAT | CCGGRTGAT | CCGGRTGAT | CCGGATGAT |
| scaffold_6 | 401 214 | CAACGGTTC | CAACGGTTC | CAACGGTTC | CAACRGTTC |
| scaffold_6 | 501 533 | GCCTCCTTG | GCCTYSTTG | GCCTYSTTG | GCCTTGTTG |
| scaffold_6 | 602 060 | CTCGCGGAC | CTCGCGGAC | CTCGCGGAC | CTCGTGGAC |
| scaffold_6 | 705 053 | CAATCAACG | CAATCAACG | CAATCAACG | CAATSAASG |
| scaffold_6 | 812 235 | AGTACGTCA | AGTAYGTCA | AGTAYGTCA | AGTATGTCA |
| scaffold_6 | 910 395 | GCCAACAAA | GCCARCAAA | GCCARCAAA | GCCAGCAAA |
| scaffold_6 | 1 000 140 | CGGCATCTC | CGGCRTCTC | CGGCRTCTC | CGGCATCTC |
| scaffold_6 | 1 100 090 | CGACATTGA | CGACRTTGA | CGACRTTGA | CGACATTGA |
| scaffold_6 | 1 200 043 | AATTTGTAA | AATTYGTAA | AATTYGTAA | AATTTGTAA |
| scaffold_6 | 1 300 008 | CTGTGGATT | CTGTRGATT | CTGTRGATT | CTGTGGATT |
| scaffold_6 | 1 400 057 | GCATGAAGG | GCATRAAGG | GCATRAAGG | GCATGAAGG |
| scaffold_6 | 1 500 031 | TCGATGTTG | TCGAYGTTG | TCGAYGTTG | TCGATGTTG |
| scaffold_6 | 1 600 014 | GCCGAGCAC | GCCGRGYAC | GCCGRGYAC | GCCGAGCAC |
| scaffold_6 | 1 750 012 | CTTGGAAGG | CTTGRAAGG | CTTGRAAGG | CTTGGAAGG |
| scaffold_6 | 1 830 280 | TGAAGTTTG | TGAARTTTG | TGAARTTTG | TGAAGTTTG |
| scaffold_6 | 1 900 029 | CTTTCCTTT | CTTTYCTTT | CTTTYCTTT | CTTTCCTTT |
| scaffold_6 | 1 950 025 | GTGGGGGAT | GTGGSGGRT | GTGGSGGRT | GTGGGGGAT |
| scaffold_6 | 2 000 142 | CGTCGTGCC | CGTCSTGCC | CGTCSTGCC | CGTCCTGCC |
| scaffold_6 | 2 100 113 | CAAGCCGCT | CAAGYCGCT | CAAGYCGCT | CAAGTCGCT |
| scaffold_6 | 2 200 135 | TCAGTGATG | TCAGWGATG | TCAGWGATG | TCAGTGATG |
| scaffold_6 | 2 300 032 | TACGCTGGC | TACGYTGGC | TACGYTGGC | TACGTTGGC |
| scaffold_6 | 2 453 019 | ACCGACGGA | ACCGRCRGA | ACCGRCRGA | ACCGGCAGA |
| scaffold_6 | 2 550 600 | CCACGCCAT | CCACRCCAT | CCACRCCAT | CCACGCCAT |
| scaffold_6 | 2 651 795 | GTCCGCGAG | GTCCACGAG | GTCCACGAG | GTCCRCGAG |
| scaffold_7 | 79 999 | ATTTCGGGA | ATTTCGGGA | ATTTCGGGA | ATTTYGGGA |
| scaffold_7 | 149 936 | GTGCGCGAT | GTGCGCGAT | GTGCGCGAT | GTGCRCGAT |
| scaffold_7 | 205 610 | TGGGTGTAC | TGGGYGTAC | TGGGYGTAC | TGGGTGTAC |
| scaffold_7 | 300 008 | TTCC---CG | TTCCcgaCG | TTCCcgaCG | TTCC---CG |
| scaffold_7 | 400 168 | ATGAATTAA | ATGARTTAA | ATGARTTAA | ATGAATTAA |
| scaffold_7 | 500 025 | ACTCGTTGC | ACTCRTTGC | ACTCRTTGC | ACTCGTTGC |
| scaffold_7 | 606 538 | CTATTCCTC | CTATTCCTC | CTATTCCTC | CTATYCCTC |
| scaffold_7 | 760 725 | AATTAATAT | AATTAATAT | AATTAATAT | AATTMATAT |
| scaffold_7 | 878 852 | TTACATCTT | TTACATCTT | TTACATCTT | TTACRTCTT |
| scaffold_7 | 969 639 | CCCACCCAC | CCCACCCAC | CCCACCCAC | CCCAYCCAC |
| scaffold_7 | 1 009 522 | GCACACTGT | GCACRCTGT | GCACRCTGT | GCACGCTGT |
| scaffold_7 | 1 099 999 | GGAAGTTGG | GGAARTTGG | GGAARTTGG | GGAAATTGG |
| scaffold_7 | 1 200 198 | TGACATCGG | TGACRTCGG | TGACRTCGG | TGACGTCGG |
| scaffold_7 | 1 300 159 | TCAACTTGG | TCAAYYTGG | TCAAYYTGG | TCAATCTGG |
| scaffold_7 | 1 376 844 | AAGCAGTAA | AAGCRGTGA | AAGCRGTGA | AAGCGGTGA |
| scaffold_7 | 1 471 380 | CCAGCGCAC | CCAGCGCAC | CCAGCGCAC | CCAGSGCAC |
| scaffold_7 | 1 652 900 | TTCAGGAAC | TTCAGGAAC | TTCAGGAAC | TTCARGAAC |
| scaffold_7 | 1 757 509 | GACGGCAAT | GACGKCAAT | GACGKCAAT | GACGGCAAT |
| scaffold_7 | 1 900 038 | TGGCGTGGA | TGGCGTRGA | TGGCGTRGA | TGGCSTRGA |
| scaffold_7 | 1 989 591 | CCGATCGAG | CCGATCGAG | CCGATCGAG | CCGAYCGAG |
| scaffold_7 | 2 018 475 | GATGCGCTC | GATGCGCTC | GATGCGCTC | GATGYGCTC |
| scaffold_7 | 2 153 444 | TGATACCAT | TGATACCAT | TGATACCAT | TGATMCCAT |
| scaffold_7 | 2 262 195 | GAGACTTTC | GAGACTTTC | GAGACTTTC | GAGAYTTTC |
| scaffold_7 | 2 355 772 | TGCCAGCGA | TGCCAGCGA | TGCCAGCGA | TGCCRGCGA |
| scaffold_7 | 2 516 731 | AGGTCGTCC | AGGTCGTCC | AGGTCGTCC | AGGTYGTCC |
| scaffold_7 | 2 655 618 | AATAA--AA | AATAA--AA | AATAAagAR | AATAAagAA |
| scaffold_7 | 2 655 728 | ACTCATTTT | ACTCATTTT | ACTCATTTT | ACTCRTTTT |
| scaffold_7 | 2 759 622 | TTGTGACGC | TTGTRACGC | TTGTRACGC | TTGTAACGC |
| scaffold_7 | 2 890 243 | CGGGATGCG | CGGGATGCG | CGGGATGCG | CGGGGTGCG |
| scaffold_7 | 2 950 028 | ACCCATACA | ACCCRTACA | ACCCRTACA | ACCCGTACA |
| scaffold_7 | 3 050 201 | CCGAGACGA | CCGAGACGA | CCGAGACGA | CCGARACGA |
| scaffold_7 | 3 150 325 | TACATGGAG | TACAYGSAG | TACAYGSAG | TACACGCAG |
| scaffold_7 | 3 247 432 | GAACGCTCA | GAACGCTCA | GAACGCTCA | GAACRCTCA |
| scaffold_7 | 3 252 776 | GAAGCGTTT | GAAGCGTTT | GAAGCGTTT | GAAGYGTTT |
| scaffold_8 | 100 122 | AGAAGAGCG | AGAAKAGCG | AGAAKAGCG | AGAATAGCG |
| scaffold_8 | 201 577 | TGCACGGTG | TGCAYGGTG | TGCAYGGTG | TGCACGGTG |
| scaffold_8 | 304 125 | TTCTGGGGA | TTCTRGGGA | TTCTRGGGA | TTCTGGGGA |
| scaffold_8 | 372 048 | TTGCGTACC | TTGCATACC | TTGCATACC | TTGCGTACC |
| scaffold_8 | 400 002 | AGGCGAGCC | AGGCRAGCC | AGGCRAGCC | AGGCGAGCC |
| scaffold_8 | 500 058 | TCGGATGCT | TCGGRTGCT | TCGGRTGCT | TCGGATGCT |
| scaffold_8 | 600 054 | TCTGGGGAG | TCTGRGGAG | TCTGRGGAG | TCTGAGGAG |
| scaffold_8 | 700 049 | TCTTAGAGC | TCTTAGAGC | TCTTAGAGC | TCTTRGAGC |
| scaffold_8 | 820 006 | CAGGGAGTT | CAGGRAGTT | CAGGRAGTT | CAGGGAGTT |
| scaffold_8 | 950 004 | TTTC----- | TTTCaactg | TTTCaactg | TTTC----- |
| scaffold_8 | 1 050 001 | TGCAAAAAC | TGCARAAAS | TGCARAAAS | TGCAAAAAC |
| scaffold_8 | 1 096 526 | CACCTCCTC | CACCTCCTC | CACCTCCTC | CACCWCCTC |
| scaffold_8 | 1 174 950 | AGTTTAGCA | AGTTTAGCA | AGTTTAGCA | AGTTYAGCA |
| scaffold_8 | 1 250 040 | GATCGGGCC | GATCRGGCC | GATCRGGCC | GATCGGGCC |
| scaffold_8 | 1 350 024 | GATATGCTC | GATACGCTC | GATACGCTC | GATAYGCTC |
| scaffold_8 | 1 450 028 | ATTCAAAGG | ATTCRAAGG | ATTCRAAGG | ATTCAAAGG |
| scaffold_8 | 1 554 996 | CAATTCAAC | CAATYCAAM | CAATYCAAM | CAATTCAAC |
| scaffold_8 | 1 699 991 | TCTGCGAAT | TCTGYGAAT | TCTGYGAAT | TCTGCGAAT |
| scaffold_8 | 1 800 057 | ACTTGAAGC | ACTTRAAGC | ACTTRAAGC | ACTTGAAGC |
| scaffold_8 | 1 900 009 | GAGCACTGG | GAGCRCTGG | GAGCRCTGG | GAGCACTGG |
| scaffold_8 | 2 050 011 | TAAGCACGC | TAAGCACGC | TAAGCACGC | TAAGMACGC |
| scaffold_8 | 2 100 043 | TACCTGTGT | TACCCGTGT | TACCCGTGT | TACCYGTGT |
| scaffold_8 | 2 200 060 | CCTCGCCTG | CCTCACTTG | CCTCACTTG | CCTCRCYTG |
| scaffold_8 | 2 300 038 | GCGAAGCTG | GCGAGGCTG | GCGAGGCTG | GCGARGCTG |
| scaffold_8 | 2 401 147 | GAATTCTGA | GAATCCTGA | GAATCCTGA | GAATYCTGA |
| scaffold_8 | 2 500 431 | TCTATTTTG | TCTAATTTG | TCTAATTTG | TCTAWTTTG |
| scaffold_8 | 2 600 536 | TATAATCTA | TATAGTCTA | TATAGTCTA | TATARTCTA |
| scaffold_8 | 2 706 410 | GTCGTTCCT | GTCGTTCCT | GTCGTTCCT | GTCGKTCCT |
| scaffold_9 | 100 121 | TGGCGGGAC | TGGCRRRAC | TGGCRRRAC | TGGCAAGAC |
| scaffold_9 | 216 066 | TGTCATAAT | TGTCATAAT | TGTCATAAT | TGTCWTAAT |
| scaffold_9 | 325 155 | CCTCGCCTA | CCTCCCCTA | CCTCCCCTA | CCTCSCCTA |
| scaffold_9 | 410 772 | CACGAGAGA | CACGAGAGA | CACGAGAGA | CACGRGAGA |
| scaffold_9 | 550 009 | CCGCCGGGA | CCGCYGGGA | CCGCYGGGA | CCGCCGGGA |
| scaffold_9 | 650 672 | GTGTTCATG | GTGTTCATG | GTGTTCATG | GTGTYCATG |
| scaffold_9 | 750 219 | GATAGCGTT | GATAACGTT | GATAACGTT | GATARCGTT |
| scaffold_9 | 750 253 | AAACCCATG | AAACCCATG | AAACCCATG | AAACYCATG |
| scaffold_9 | 850 036 | CGAGCTCGG | CGAGCTCGG | CGAGCTCGG | CGAGYTCGG |
| scaffold_9 | 951 067 | ACCTCCCTG | ACCTTCCTG | ACCTTCCTG | ACCTYCCTG |
| scaffold_9 | 1 050 035 | GGGGTCATT | GGGGYCATT | GGGGYCATT | GGGGTCATT |
| scaffold_9 | 1 150 022 | CGTTCGTCA | CGTTCGTCA | CGTTCGTCA | CGTTYGTCA |
| scaffold_9 | 1 250 027 | TATGCTAGC | TATGSYAGC | TATGSYAGC | TATGCTAGC |
| scaffold_9 | 1 350 023 | CTGCGGCGA | CTGCKGCSA | CTGCKGCSA | CTGCGGCGA |
| scaffold_9 | 1 450 046 | CAGAATGAT | CAGARTGAT | CAGARTGAT | CAGAATGAT |
| scaffold_9 | 1 550 112 | GCGTCGATT | GCGTYGATT | GCGTYGATT | GCGTTGATT |
| scaffold_9 | 1 651 678 | GAGTAGTCG | GAGTAGTCG | GAGTAGTCG | GAGTRGTCG |
| scaffold_9 | 1 765 766 | CCTGCAACC | CCTGCAACC | CCTGCAACC | CCTGYAACM |
| scaffold_9 | 1 863 807 | TCAACGGTA | TCAACGGTA | TCAACGGTA | TCAAYGGTA |
| scaffold_10 | 71 418 | CAGTATGCC | CAGTATGCC | CAGTATGCC | CAGTRTGCC |
| scaffold_10 | 100 951 | TCACGAGTC | TCACGAGTC | TCACGAGTC | TCACRAGTC |
| scaffold_10 | 200 107 | TCTGTGATG | TCTGCGATG | TCTGCGATG | TCTGYGATG |
| scaffold_10 | 310 393 | AGTTTAAAT | AGTTCAAAT | AGTTCAAAT | AGTTYAAAT |
| scaffold_10 | 400 433 | GCATAGAAT | GCATGGAAT | GCATGGAAT | GCATRGAAT |
| scaffold_10 | 500 060 | TTCGTCGTG | TTCGCCGTG | TTCGCCGTG | TTCGYCGTG |
| scaffold_10 | 600 762 | GCACGGACC | GCACAGACC | GCACAGACC | GCACRGACC |
| scaffold_10 | 601 183 | GAGGTATAC | GAGGTATAC | GAGGTATAC | GAGGKATAC |
| scaffold_10 | 700 186 | GCACGGTCT | GCACAGTCT | GCACAGTCT | GCACRGTCT |
| scaffold_10 | 800 334 | AGGCACATA | AGGCGCATA | AGGCGCATA | AGGCRCATA |
| scaffold_10 | 900 489 | ATTCAATTG | ATTCWATTG | ATTCWATTG | ATTCRATTG |
| scaffold_10 | 1 001 291 | TGGAGCATC | TGGACCATC | TGGACCATC | TGGASCATC |
| scaffold_10 | 1 101 282 | CGACCCCAA | CGACYCCAA | CGACYCCAA | CGACTCCAA |
| scaffold_10 | 1 200 070 | CCCACCACC | CCCAYCACC | CCCAYCACC | CCCATCACC |
| scaffold_10 | 1 300 202 | CCCCGCTGC | CCCCRCTGC | CCCCRCTGC | CCCCACTGC |
| scaffold_10 | 1 400 004 | GCATGCGAT | GCATKCGAT | GCATKCGAT | GCATTCGAT |
| scaffold_10 | 1 470 370 | GGGTGTTTA | GGGTGTTTA | GGGTGTTTA | GGGTRTTTA |
| scaffold_11 | 24 406 | GAGCGAGGA | GAGCGAGGA | GAGCGAGGA | GAGCRAGGA |
| scaffold_11 | 95 561 | ATGAGTCCC | ATGACTCCC | ATGACTCCC | ATGAMTCCC |
| scaffold_11 | 191 249 | CTATCACGT | CTATCACGT | CTATCACGT | CTATSACGT |
| scaffold_11 | 264 287 | TAGCGCAAA | TAGCGCAAA | TAGCGCAAA | TAGCRCAAA |
| scaffold_11 | 308 590 | AGCTCAGGT | AGCTCAGGT | AGCTCAGGT | AGCTYAGGT |
| scaffold_11 | 389 586 | CACTGAGAG | CACTGAGAG | CACTGAGAG | CACTSAGAG |
| scaffold_11 | 430 276 | CTTCGCATC | CTTCGCATC | CTTCGCATC | CTTCKCATC |
| scaffold_11 | 463 317 | CTTGCGGCT | CTTGCGGCT | CTTGCGGCT | CTTGYGGCT |
| scaffold_11 | 542 025 | TCTCTCCCT | TCTCTCCCT | TCTCTCCCT | TCTCYCCCT |
| scaffold_11 | 599 846 | TTGTAAAAT | TTGTAAAAT | TTGTAAAAT | TTGTRAAAT |
| scaffold_11 | 678 225 | CTCAATGGG | CTCAATGGG | CTCAATGGG | CTCARTGGG |
| scaffold_11 | 721 025 | CGCGGCCCA | CGCGGCCCA | CGCGGCCCA | CGCGSCCCA |
| scaffold_11 | 799 972 | AAACGGATT | AAACGGATT | AAACGGATT | AAACRGATT |
| scaffold_11 | 867 020 | ATGCAGAGG | ATGYAGAGG | ATGYAGAGG | ATGYMGAGG |
| scaffold_11 | 882 149 | AATGCGACA | AATGCGACA | AATGCGACA | AATGYGACA |
| scaffold_11 | 987 108 | TTCTGGCTT | TTCTGGCTT | TTCTGGCTT | TTCTRGCTT |
| scaffold_11 | 1 134 373 | ATGTAAAAA | ATGT-AAAA | ATGT-AAAA | ATGTaAAAA |
| scaffold_11 | 1 207 025 | CACAGTTAT | CACAGTTAT | CACAGTTAT | CACARTTAT |
| scaffold_11 | 1 299 971 | AAGA-AATG | AAGA-AATG | AAGA-AATG | AAGW-RATG |
| scaffold_11 | 1 400 060 | AGTACACAG | AGTASACAG | AGTASACAG | AGTAGACAG |
| scaffold_11 | 1 500 691 | GTTCGAACG | GTTCRAACG | GTTCRAACG | GTTCGAACG |
| scaffold_11 | 1 648 648 | TGGTACATC | TGGTRCATC | TGGTACATC | TGGTGCATC |
| scaffold_11 | 1 799 978 | CCCGTACTC | CCCGCACAC | CCCGCACAC | CCCGYACWC |
| scaffold_11 | 1 860 242 | CGACATCCT | CGACATCCT | CGACATCCT | CGACWTCCT |
| scaffold_11 | 1 863 549 | GTATGCAGGC | GTATGCAGGC | GTATGCAGGC | GTAYGCASGC |
| scaffold_11 | 1 989 559 | AGTTGCCCA | AGTTGCCCA | AGTTGCCCA | AGTTRCCCA |
| scaffold_11 | 2 101 983 | GCTGTGCTG | GCTGYGCTG | GCTGYGCTG | GCTGCRCTG |
| scaffold_11 | 2 102 188 | TCATCGTCA | TCATCGTCA | TCATCGTCA | TCATYGTCA |
| scaffold_11 | 2 319 374 | GATGGTATG | GATGGTATG | GATGGTATG | GATGRTATG |
| scaffold_12 | 7 413 | CGCTGCGCT | CGCTGCGCT | CGCTGCGCT | CGCTRCGCT |
| scaffold_12 | 38 692 | GGGGTCCGA | GGGGTCCGA | GGGGTCCGA | GGGGYCCGA |
| scaffold_12 | 61 918 | TGCGCCGTG | TGCGCCGTG | TGCGCCGTG | TGCGYCGTG |
| scaffold_12 | 130 671 | ATTGGCAGC | ATTGGCAGC | ATTGGCAGC | ATTGRCARC |
| scaffold_12 | 193 854 | TTCTCCTTG | TTCTCCTTG | TTCTCCTTG | TTCTSCTTG |
| scaffold_12 | 238 296 | AGCCGAGTG | AGCCGAGTG | AGCCGAGTG | AGCCRAGTG |
| scaffold_12 | 270 146 | AGTATGCCT | AGTAYGCCT | AGTAYGCCT | AGTAYRCCT |
| scaffold_12 | 273 774 | AGGATGAGG | AGGATGAGG | AGGATGAGG | AGGAYGAGG |
| scaffold_12 | 275 215 | CTACACAGG | CTACACAGG | CTACACAGG | CTACRCAGG |

Table A comprises sets of SNP markers described as 9-mers. Positional information refers to the 11 out 12 substantial contigs of the PC15 genome sequence assembly (JGI). Because a dikaryon incorporates two sets of chromosomes, one from each haploid parent, there are two allelic copies (two characters or elements of the genotype) at each marker locus for FiBABH-334. The IUPAC nucleotide and so-called “ambiguity” codes (also see Annex C, Appendix 2, Table 1, Nucleotide and Amino Acid Symbols as set forth in Standard ST. 25 of the Handbook on Industrial Property Information and Documentation (WIPO) (December 2009)) which are actually heteroallelism codes when used to represent a dikaryon or diploid genotype, are used in Table A to represent heteroallelic DNA sequence positions, wherein each of two alleles incorporates a different nucleotide at a particular position, in the observed 9-base DNA marker sequences reported above, each of which represents a genotypic marker locus. The identity of each marker locus is specified by the scaffold and SNP position information derived from the PC15 V2.0 standard reference genome sequence published by the U.S. Department of Energy Joint Genome Institute (Alfaro et al. 2016), incorporated herein by reference.

It will be appreciated however that any pair of suitable Polymerase Chain Reaction (PCR) primers that bracket the defined marker regions may be used for identifying the alleles, using methods of designing and using suitable PCR primers that are well known in the art. Methods employing these and other markers to determine genealogical relationships between cultures are provided below.

Mushroom-forming fungi exhibit an alternation of generations, from dikaryotic (N+N, with two haploid nuclei, functionally like the 2N diploid state) to monokaryotic (1N) and further upon mating to become dikaryotic again. In most eukaryotes, a parent is conventionally considered to be either diploid or dikaryotic. The haploid ‘generation’ is often, but not always, termed a gamete (e.g., pollen, sperm). In fungi, which are microorganisms, the haploid generation can live and grow indefinitely and independently, for example in laboratory cell culture; while these haploid monokaryons function as gametes in matings, they are equivalent to inbred lines (e.g., of plants) and are more easily referred to as lines (or ‘monokaryon-parents’ of hybrids). Herein, the standalone term ‘parent’ refers, depending on context, to the dikaryotic culture that is either a, or the, direct progenitor of a haploid line culture, or else the progenitor-once-removed of a strain belonging to the subsequent dikaryotic generation obtained from a mating of at least one such line. The term ‘line’ thus refers narrowly to a haploid (N) homoallelic culture within the lifecycle. The N+N dikaryon resulting from a mating, or comprising a breeding stock, or comprising a culture used to produce a crop of mushrooms, may be called a ‘strain’.

In a preferred embodiment, the strain of the invention is selected from the group consisting of:
(a) F1 hybrids produced by mating a monokaryon from FiBABH-334 with a second line,
(b) the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023,
(c) derivatives of the strain (b), said derivatives being cells obtained from said FiBABH-334 strain by somatic selection, tissue culture selection, single spore germination, multiple spore germination, matings between mycelium obtained from monokaryotic or dikaryotic spores from these hybrids, selfing, repeated mating back to the initial culture, mutagenesis, trait conversion, introgressive trait conversion, inbreeding or transformation.

In some embodiments, the strain of the invention may be obtained using at least one strain development technique selected from the group consisting of: inbreeding, outbreeding, i.e, selfing, backmating, introgressive trait conversion, derivation, somatic selection, tissue selection, single-spore selection, multispore selection, pedigree-assisted breeding, marker assisted selection, mutagenesis and transformation, and applying said at least one strain development technique to a first mushroom strain, or parts thereof, said first strain comprising at least one set of chromosomes of a monokaryon of the strain FiBABH-334.

In a preferred embodiment, the strain of the invention is a sporeless Pleurotus strain of the F2 or F3 generation, descended from the strain FiBABH-334, and comprising preferably at least 90%, at least 85%, at least 80%, at least 75%, at least 70%, at least 65%, at least 60%, at least 55%, at least 50%, at least 45%, at least 40%, at least 30%, at least 20%, or at least 10%, (preferably comprising at least 99%, or at least 98%, or at least 95%, or at least 92%, or at least 90%, or at least 85%, or at least 80%, or at least 75%, or at least 70%), of the Single-Nucleotide Polymorphisms (SNPs) present in the genome of the strain FiBABH-334, a representative culture of said strain having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number 1-5939; or comprising between 80%-100%, between 60%-80%, between 40-60%, between 20-40%, between 10-20%, between 10-15%, or between 4-10% (preferably comprising between 80%-100%, or between 60-80%), of the Single-Nucleotide Polymorphisms (SNPs) present in the genome of the strain FiBABH-334, a representative culture of said strain having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number I-5939.

In another preferred embodiment, the strain of the invention descends from FiBABH-334, or from a strain derived from FiBABH-334, and comprises at least about 280, at least about 270, at least about 260, at least about 250, at least about 240, at least about 230, at least about 220, at least about 210, at least about 200, at least about 180, at least about 170, at least about 150, at least about 130, at least about 100, at least about 75, at least about 65, at least about 50, or at least about 25 allelic markers out of the 298 sequence-characterized allelic markers of FiBABH-334 listed in Table A.

In another preferred embodiment, the strain of the invention descends from FiBABH-334, or from a strain derived from FiBABH-334, comprising at least 90%, at least 85%, at least 80%, at least 75%, at least 70%, at least 65%, at least 60%, at least 55%, at least 50%, at least 45%, at least 40%, at least 30%, at least 20%, or at least 10% (preferably comprising at least 99%, or at least 98%, or at least 95%, or at least 92%, or at least 90%, or at least 85%, or at least 80%, or at least 75%, or at least 70%), of the Single-Nucleotide Polymorphisms (SNPs) present in the genome of the strain FIBABH-334, a representative culture of said strain having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number 1-5939; or comprising between 80%-100%, between 60%-80%, between 40-60%, between 20-40%, between 10-20%, between 10-15%, or between 4-10% (preferably comprising between 80%-100%, or between 60-80%), of the sequence-characterized allelic markers of FiBABH-334 listed in Table A.

In another aspect, the present invention targets a sporeless Pleurotus mushroom strain that is derived from the initial strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023,

Preferably, said derived strain comprises at least 99%, at least 98%, at least 95%, at least 92%, at least 90%, at least 85%, at least 80%, at least 75%, at least 70%, at least 65%, at least 60%, at least 55%, at least 50%, at least 45%, at least 40%, at least 30%, at least 20%, or at least 10% (preferably comprising at least 99%, or at least 98%, or at least 95%, or at least 92%, or at least 90%, or at least 85%, or at least 80%, or at least 75%, or at least 70%), of the Single-Nucleotide Polymorphisms (SNPs) present in the genome of the strain FiBABH-334, a representative culture of said strain having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number 1-5939; or comprising between 80%-100%, between 60%-80%, between 40-60%, between 20-40%, between 10-20%, between 10-15%, or between 4-10% (preferably comprising between 80%-100%, or between 60-80%), of the sequence-characterized allelic markers of FiBABH-334 listed in Table A.

The strain culture of the invention is not the parent strain TPLO4 strain. In a preferred embodiment, the strain of the invention differs from TPLO4 on at least 10%, preferably on at least 20% of its allelic markers. In other words, the strain of the invention does not have more than 90%, preferably more than 80% identity with TPLO4.

In another aspect, the present invention targets a culture of any of the mushroom strain described above.

The term “mushroom culture” herein designates the mushroom microorganism propagated on various growth media and substrates. It refers to a physical strain, line, monokaryon or dikaryon. Once obtained, a culture can be vegetatively reproduced at laboratory level.

The culture of the invention characterized in that:
(a) the total yield performance of the crops of said culture is at least equal to the yield performance of crops of the Spoppo strain of Pleurotus, and
(b) the average total height of the resulting mushrooms, including their stipe and cap, is significantly inferior to the average height of Spoppo mushrooms, and
(c) the average height of the stipe of the resulting mushrooms, is significantly inferior to the average height of Spoppo mushroom stipe, and
(d) the cap of the resulting mushrooms is flatter than the caps of Spoppo mushrooms.

In particular, the total yield performance of the culture of the invention is of about 23.26% over 2 flushes (see example below).

Moreover, the average total height of the mushroom (including cap and foot) is preferably comprised between 25 mm and 40 mm. Typically, its average total height (including cap and foot) does not exceed 50 mm for mushroom having a cap diameter not exceeding 85 mm.

### Pleurotus Mushrooms

As used herein, the term “mushroom” designates, depending on the context in which it is used, the reproductive structure of a Pleurotus fungus or a cultivated food product of the same name.

Pleurotus is a genus of mushrooms having three distinct parts:
a fleshy shell or spatula shaped cap (pileus),
a short or long lateral or central stalk called stipe
long ridges and furrows underneath the pileus, called gills or lamellae. The gills stretch from the edge of the cap down to the stalk and bear the spores.

“Spores” are the reproductive propagule of the mushroom. Living spores are either dikaryons or monokaryons in a dormant state. Other parts of the mushroom include caps, stipes, gills, cells (defined as hyphal compartments incorporating nuclei, mitochondria, cytoplasm, a cell membrane and a cell wall including cross-walls), hyphae and mycelium.

As used herein, the term “monokaryon” designates a haploid culture with a single type (or somatic lineage) of haploid nucleus (cytogenetically represented as n). It is ordinarily reproductively incompetent (i.e. does not fruit) but may function as a gamete in sexually complementary anastomoses. Though haploid, monokaryons are also referred to as “lines” which transmit a uniform genotype to offspring.

On the other hand, “dikaryon” refers to a culture which has two complementary types of haploid nuclei in a common cytoplasm. It is thus functionally and physiologically analogous to a diploid individual (but cytogenetically represented as n+n rather than 2n). Unlike monokaryons, it is reproductively competent (i.e. able to fruit). Dikaryons are also called “strains” in the breeding context. In the context of the invention dikaryotic strains produced from controlled matings are also called “hybrids”.

As used herein the term “anastomosis” designates the fusion of two or more hyphae that achieves cytoplasmic continuity. Moreover the term “plasmogamy” refers to the establishment, via anastomosis, of cytoplasmic continuity leading to the formation of a sexual dikaryon with exchange of nuclei, mitochondria, cytoplasm and other intracellular components. The sexual union of two cultures via anastomosis and plasmogamy is herein referred to as “mating”.

### Pleurotus Breeding

As other fungal specie, Pleurotus has different reproduction processes: vegetative reproduction (mitosis), sexual reproduction (meiosis) and parasexual reproduction (other phenomena). Pleurotus show the typical life cycle of Basidiomycete fungi. It will start with the germination of spores that will conduct to a monokaryotic mycelium (N). When two compatible monokaryotic mycelium (different loci Mat A and Mat B) are confronted, a fertile dikaryotic mycelium (N+N) is created by hyphal fusion or plasmogamy.

### Growing Process of Pleurotus

In one further aspect, the present invention concerns a mushroom produced by growing a crop of mushrooms from the strain of the invention, or from the culture of the invention. Mushroom parts, and mushroom pieces, are also encompassed.

The invention also concerns a product incorporating the strain of the invention or the culture of the invention, including spawn, inoculum, processed foods, colonised substrates including grain, substrate and friable particulate matter.

Also, the present invention is drawn to a method for producing mushrooms, said method comprising:
a) Inoculating a mushroom growth medium with a sporeless Pleurotus strain of the invention or with a culture of the invention,
b) Maintaining said inoculated growth medium under conditions conductive to mushroom fruiting and
c) Collecting mushrooms from said growth medium.

Pleurotus can be grown on substrate prepared from agricultural by-products or waste (e.g. straw)

The selection of appropriate substrate and spawn run time can be easily done by the skilled person, given its simplicity in large-scale cultivation. Broadleaf, hardwood, saw-dust, and straw-based substrates with added supplements are more often used in commercial production. The artificial substrates must be pre-treated in a clean environment, mainly to eliminate contaminants. Pleurotus species can colonize and produce mushrooms on pretreated conifer (Pinus species) wood chips. However, the non-pretreated confer wood chips substrate inhibits the mycelial colonization presence of inhibitory components. Pleurotus species can also utilize wood waste or unused wood residues to promote economic growth and rescue the forest ecosystem.

Spawn is a mushroom seed, used to propagate the mushroom mycelia in the desired solid substrate during cultivation. The temperature of the spawn room is preferably maintained between 25 and 30° C. The spawn run period and primordial initiation are generally observed during 24-30 days. Light is not a prerequisite in the spawn running room. Spawning is done in a cleaned spawning area in the bulk chamber. Preparation of Pleurotus spawn is described e.g., in Raman et al, 2021.

Pleurotus is a micro-organism that can be handled by classical microbiology techniques in laboratories under sterile conditions (laminar hood and/or clean area, sterile media, instruments, equipment, packages etc.). Its optimum conditions are temperature approximately 24° C., pH approximately 7. Mycelium or spores can be stored in a cool room (e.g. 2 to 4° C.). Growing media can be malt extract agar, potato dextrose agar, potato dextrose yeast agar, specific Pleurotus substrate agar, etc.

The seeded substrates, broth, stalk, and stick (mycelia bit/through spawning) can be inoculated in the cultivation substrates (perforated polythene bags, polypropylene bottles, and trays). Different types of cultivated methods are employed in Pleurotus production, for instance, wall-frame, shelf, tray, jar, bag, bottle, and grid-frame methods. The most practiced method includes shelf cultivation, a bag, and a bottle. The shelf (row/bed) cultivation method is considered as a low cost and high production technique. In the bag cultivation method, the spawn is tied on the top and is placed horizontally in rows. The advantages of plastic bottle cultivation are high yield and efficiency with limited land space.

Cultivation bags and bottles are placed vertically or horizontally for spawn running, and the spawn incubation chamber are preferably maintained at 24-25° C. and 90% RH. The Pleurotus species spawn run takes about 12-26 days at 24±1° C. in the substrate. The spawn inoculated bags/bottles are allowed to colonize (outside becoming white), and they are finally transferred into the cultivation chamber. The chamber are maintained at 15-23° C., relative humidity at 85-95%, and the photoperiod can be of 12 h/day with a light density at 15-350 lux for commercial production. Substrate supplementation with an external source of nitrogen is recommended for enhancing the oyster mushroom yield of Pleurotus species. Pleurotus species cultivation has been tested in different bag-ging systems. Plastic bags are found to yield higher harvest than other methods like trays, racks, and cylindrical containers bagging systems. The plastic bags hanged in rows can reduce the contamination level and allow good air circulation (Raman et al, 2021).

Primordial initiation is generally observed from 16 to 27 days. Generally, mature mushrooms are visible within 3-4 days after pinhead formation. The mature mushrooms are then harvested before spraying water. The entire cropping can be completed in 50-55 days.

After picking or harvesting other mushrooms start growing as successive groups at approximately weekly intervals. The period of mushroom production within a cropping cycle, separated by intervals of non-production is herein called a “flush”. Successive flushes generally produce decreasing yield values. Two or three flushes are usual nowadays in industrial systems, more in traditional ones.

Finally the crop is removed, spent substrate is discarded, the facility cleaned and a new crop cycle commences.

Crop yield is herein defined as the mushroom weight picked (or harvested) per weight picked (or harvested) per weight of pasteurised substrate i.e. as a percentage.

Mushrooms Processing after Harvesting

For the fresh market, mushrooms are hand-picked. A harvest period over 3 to 5 days (staggering) is needed for hand picking. This is obtained by respectively a smooth transition from vegetative to reproductive phase, or a quicker transition, which has an impact on the number and correlatively on the size of mushrooms.

Fresh mushrooms are very perishable and can be preserved only if adequately processed. The decrease of quality and loss of value during the storage period are mainly due to weight loss, shrinkage, browning and spore formation. Postharvest techniques enhance shelf life, and mushroom quality plays a vital role in commercialization.

The shelf-life of freshly harvested Pleurotus mushroom is reported as 8-14 days at 0° C. or 1-2 days at 20° C. Controlled packing is an alternative technique for extending mushrooms shelf-life without chemical treatment (low temperature, modified atmosphere packing . . . ).

### Development of Progenies

In one further aspect, the present invention concerns cells, spores, hyphae and mycelium obtained from the strain of the invention, or from the culture of the invention.

It also concerns monokaryons of the strains of the invention. Indeed, even if the strain of the invention is characterized as being “sporeless”, they produce some spores. These very few spores can be isolated on spores print (never above 50 spores, preferably 30 spores, for a whole spore print during 24 hours), monokaryons from the strain of the invention can also isolated by protoplasting or dedikaryotization. The SNPs present in the genome of the monokaryons of the invention can be easily identified by whole genome sequencing or by using conventional markers such as those described in U.S. Pat. No. 7,608,760 or 9,017,988. Table A gives a number of useful sequences that can therefore characterize the monokaryons of the invention. Any other SNP can however be used to identify the monokaryons of the invention. In a preferred embodiment, the monokaryon of the invention descends from the strain FiBABH-334 and contains at least 90%, at least 85%, at least 80%, at least 75%, at least 70%, at least 65%, at least 60%, at least 55%, at least 50%, at least 45%, at least 40%, at least 30%, at least 20%, at least 10%, or between 80%-100%, between 60%-80%, between 40-60%, between 20-40%, between 10-20%, between 10-15%, or between 4-10% of the SNPs present in the genome of the strain FiBABH-334, preferably of the SNPs disclosed in Table A. In another preferred embodiment, the monokaryon of the invention descends from the strain FiBABH-334 and contains at least about 280, at least about 270, at least about 260, at least about 250, at least about 240, at least about 230, at least about 220, at least about 210, at least about 200, at least about 180, at least about 170, at least about 150, at least about 130, at least about 100, at least about 75, at least about 65, at least about 50, or at least about 25 allelic markers out of the 298 sequence-characterized allelic markers of the strain FiBABH-334 listed in Table A.

To calculate the percentage of SNPs between two strains, one can compare the composite 9-mer genotype at each locus and assign a value if 1 for a perfect match, or a 0 for anything less than a perfect match. Then the values can be totaled for all loci in each pairwise comparison between strains, and divided by the total number of loci compared. The resulting decimal can be eventually converted to %.

The term “approximately” or “about” herein inculcates a range of plus or minus 10% above or below the stated value.

In another embodiment, the culture of the invention comprises at least one set of chromosomes having at least 65%, 70%, 75%, 85%, 90%, 95%, 96%, 97%, 98%, or 99% genetic identity with the chromosomes of the monokaryon of the invention. In a further embodiment, the culture of the invention comprises at least one set of chromosomes having a genotype with at least 65%, 70%, 75%, 85%, 90%, 95%, 96%, 97%, 98%, or 99% representation of the markers present on the chromosomes of the monokaryon of the invention.

In a further aspect, the present invention relates to a method for developing a new sporeless Pleurotus culture, said method comprising applying at least one mushroom strain development technique to the strain FiBABH-334, a representative culture of which having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number 1-5939, or to a progeny thereof, to provide a new culture.

Also, the present invention relates to a method for developing a new sporeless Pleurotus culture, said method comprising applying at least one mushroom strain development technique to the monokaryon of the invention, to provide a new culture.

In particular, said method can comprise mating the monokaryon of the invention or the strain of the invention with a second Pleurotus monokaryon or dikaryon.

Preferably, said new culture will have any of the features described above for the strains of the invention. Specifically, said new culture will preferably have any of the following desired traits:
(a) the total yield performance of the crops of said culture is at least equal to the yield performance of crops of the Spoppo strain of Pleurotus, and
(b) the average total height of the resulting mushrooms, including their stipe and cap, is significantly inferior to the average height of Spoppo mushrooms, and
(c) the average height of the stems of the resulting mushrooms, is significantly inferior to the average height of Spoppo mushroom stems, and
(d) the cap of the resulting mushrooms is flatter than the caps of Spoppo mushrooms.

These features have been described in details above. The strain Spoppo is described notably in the plant patent application US2006/0010549.

In a particularly preferred embodiment, this new culture will be the F1, F2, F3, or F4 generation descended from the strain FiBABH-334. As such, it may comprise at least 90%, at least 85%, at least 80%, at least 75%, at least 70%, at least 65%, at least 60%, at least 55%, at least 50%, at least 45%, at least 40%, at least 30%, at least 20%, at least 10%, or between 80%-100%, between 60%-80%, between 40-60%, between 20-40%, between 10-20%, between 10-15%, or between 4-10% of the Single-Nucleotide Polymorphisms (SNPs) present in the genome of the strain FiBABH-334, preferably at least 90%, at least 85%, at least 80%, at least 75%, at least 70%, at least 65%, at least 60% of the sequence-characterized allelic markers of FiBABH-334 listed in Table A.

In another preferred embodiment, this new culture contains at least about 280, at least about 270, at least about 260, at least about 250, at least about 240, at least about 230, at least about 220, at least about 210, at least about 200, at least about 180, at least about 170, at least about 150, at least about 130, at least about 100, at least about 75, at least about 65, at least about 50, or at least about 25 out of the 298 sequence-characterized allelic markers of FiBABH-334 listed in Table A.

In another aspect, the present invention relates to a method of producing a mushroom culture comprising the steps of:
(a) growing a progeny culture produced by mating the culture of the invention with a second Pleurotus culture;
(b) mating the progeny culture with itself or with a different culture to produce a progeny culture of a subsequent generation;
(c) growing a progeny culture of a subsequent generation and mating the progeny culture of a subsequent generation with itself or with a different culture; and
(d) repeating steps (b) and (c) for an additional 0-5 generations to produce a mushroom culture.

In a particular embodiment, said method comprises the steps of:
(a) obtaining a molecular marker profile of the strain FiBABH-334;
(b) obtaining an F1 hybrid culture comprising at least one set of chromosomes of the strain FiBABH-334;
(c) mating a culture obtained from the F1 hybrid culture (b) with a different mushroom culture; and
(d) selecting progeny that possess characteristics of said molecular marker profile of the strain FIBABH-334.

### Definitions

In order to provide clear and consistent understanding of the specification and claims, including the scope to be given such terms, the following definitions are provided.

Allele: one of two or more alternative forms of a gene that arise by mutation and are found at the same place on a chromosome; a heritable unit of the genome at a defined locus, ultimately identified by its DNA sequence (or by other means).

Anastomosis: Fusion of two or more hyphae that achieves cytoplasmic continuity.

Basidiomycete: A monophyletic group of fungi producing meiospores on basidia; a member of a corresponding subdivision of Fungi such as the Basidiomycetales or Basidiomycotina.

Basidium: The meiosporangial cell, in which karyogamy and meiosis occur, and upon which the basidiospores are formed.

Bioefficiency: For mushroom crops, the net fresh weight of the harvested crop divided by the dry weight of the compost substrate at the time of spawning, for any given sampled crop area or compost weight.

Breeding: Development of strains, lines or cultures using methods that emphasize sexual mating.

Cap: Pileus; part of the mushroom, the gill-bearing structure.

Carrier substrate: A medium having both nutritional and physical properties suitable for achieving both growth and dispersal of a culture.

Cloning: Somatic propagation without selection; produces a clone, which is one category of genealogical relationship (i.e., ‘identity’).

Combining ability: The capacity of an individual to transmit superior performance to its offspring. General combining ability is an average performance of an individual in a particular series of matings.

Culture: The tangible living organism; the organism propagated on various growth media and substrates; a portion of, or the entirety of, one physical strain, line, monokaryon or dikaryon; the sum of all of the parts of the culture, including hyphae, mushrooms, spores, cells, protoplasts, nuclei, mitochondria, cytoplasm, DNA, RNA, and proteins, cell membranes and cell walls.

Dedikaryotization: Conversion of a dikaryon into a pair of a monokaryon.

Derivation: Development of strains, lines or cultures generally using methods other than sexual mating, and/or undertake development solely or predominantly from an initial strain or culture; see Derived strain, Derived culture.

Derived culture: A culture obtained by derivation as defined above, exemplified by but not restricted to ‘derived strain’ or ‘derived line’; one category of genealogical relationship.

Derived lineage group: The set of strains or cultures derived solely from a single initial strain or culture (which is the earliest member of the group).

Derived strain/line: A strain/line developed solely or predominantly from a single initial strain/line. Methods employed to obtain derived strains/lines from an initial strain/line include somatic selection, tissue culture selection, single-spore germination, multiple-spore germination, selfing, repeated mating back to the initial culture, mutagenesis, and transformation, to provide some examples. In Pleurotus ostreatus, properties of derived strains include a high fidelity to the genotype and phenotype of the initial strain. In somatic selection and tissue culture selection, the derived culture may be a clone, or virtually a clone, of the initial culture, and, as with mutagenesis, it may not be feasible to specify an actual difference with the initial strain; measurable genetic identity with the initial strain may reach 100%. In a transformed-derived strain, 99.99+% of the genetic composition is that of the initial strain; the small portion of introduced DNA is ordinarily identifiable. In single-spore germinations and multiple-spore germinations, 100% of the genetic composition of the derived strain is that of the initial strain;

Descent: Genealogical descent over a limited number (e.g., 10 or fewer) of sexual generations; one category of genealogical relationship.

Dikaryon: As a term of art this refers to a sexual dikaryon: a culture which has two complementary (i.e., necessarily heteroallelic at the MAT loci) types of haploid nuclei in a common cytoplasm, and is thus functionally and physiologically analogous to a diploid individual (but cytogenetically represented as N+N rather than 2N), and which is reproductively competent (in the absence of any rare interfering genetic defects at loci other than MAT), and which exhibits vegetative incompatibility reactions with other dikaryons; also called a strain or stock in the strain development context.

Dikaryotic: Having the character of a dikaryon: two haploid nuclei in a common cytoplasm; ordinarily taken to mean two sexually complementary nuclei, but there are exceptions.

Diploid: Having two haploid chromosomal complements within a single nuclear envelope.

Directed mutagenesis: a process of altering the DNA sequence of at least one specific gene locus.

Flush: A period of mushroom production within a cropping cycle, separated by intervals of non-production; the term flush encompasses the terms ‘break’ and ‘wave’ and can be read as either of those terms.

Foot: synonymous of stem or stipe.

Fungus: A microorganism classified as a member of the Kingdom Fungi.

Genealogical relationship: A familial relationship of identity, descent, or derivation from one or more progenitors, for example that between parents and offspring.

Genetic identity: The genetic information that distinguishes an individual, including representations of said genetic information such as, and including: genotype, genotypic fingerprint, genome sequence, genetic marker profile; “genetically identical”=100% genetic identity, “X % generically identical”=having X % genetic identity, etc. % genetic similarity may be used instead of % generic identity when that percentage is less than 100.

Genotypic fingerprint: A description of the genotype at a defined set of marker loci; the known genotype.

Genetic similarity (or genotypic similarity): an expression of the degree to which one set of genetic markers, i.e., one genotype, resembles another. Any representative set of genetic markers, for example SNP markers, can be used. The proportion of markers shared in the genotypes of two individuals or cultures can be expressed to quantify the degree of resemblance between the two cultures, and is an inversely proportional measure of their distinctiveness. The terms can be used interchangeably with (percent) genetic or genotypic identity. The percentage of similarity can be based on the genotypes for any set of markers.

Gill: Lamella; part of the mushroom, the hymenophore- and basidium-bearing structure.

Haploid: Having only a single complement of nuclear chromosomes; see monokaryon.

Heteroallelic: Having two different alleles at a locus; analogous to heterozygous.

Heteroallelism: Differences between homologous chromosomes in a dikaryotic genotype; analogous to heterozygosity.

Heteromixis: Life cycle involving mating between two different non-sibling haploid individuals or gametes; outbreeding.

Homoallelic: Having not more than one allele at a locus. The equivalent term in a diploid organism is ‘homozygous’. Haploid lines are by definition entirely homoallelic at all non-duplicated loci.

Hybrid: Of biparental origin, usually applied to dikaryotic strains and cultures produced in controlled matings.

Hybridizing: Physical association, for example on a petri dish containing a sterile agar-based nutrient medium, of two cultures, usually monokaryons, in an attempt to achieve anastomosis, plasmogamy, and formation of a sexual dikaryon (=mating); succeeding in the foregoing.

Hyphae: Threadlike elements of mycelium, composed of cell-like compartments.

Inbreeding: Matings that include sibling-line matings (‘selfing’), back-matings to parent lines or strains, and intramixis; reproduction involving parents that are genetically related.

Induced mutagenesis: a non-spontaneous process of altering the DNA sequence of at least one gene locus.

Initial strain, initial culture: A strain or culture which is used as the sole or predominant starting material in a strain derivation process; more particularly a strain or culture from which a derived strain or derived culture is obtained; the earliest member of a derived lineage group.

Inoculum: A culture in a form that permits transmission and propagation of the culture, for example onto new media; specialized commercial types of inoculum include spawn and CI, wherein the culture is present on a carrier substrate.

Intramixis: A uniparental sexual life cycle involving formation of a complementary ‘mated’ pair of postmeiotic nuclei within the basidium or individual spore; superficially appears to be an asexual process.

Introgressive trait conversion: mating offspring of a hybrid to a parent line or strain such that a desired trait from one strain is introduced into a predominating genetic background of the other parent line or strain.

Lamella: see ‘gill’.

Line: A culture used in matings to produce a hybrid strain; ordinarily a monokaryon which is thus homoallelic, otherwise a non-dikaryotic culture which is highly homoallelic; practically, a functionally monokaryotic and entirely or predominantly homoallelic culture; analogous in plant breeding to an inbred line which is predominantly or entirely homozygous.

Lineage group: see ‘derived lineage group’. The set of strains or cultures derived solely from a single initial strain or culture.

Locus: A defined contiguous part of the genome, homologous although often varying among different genotypes; plural: loci.

Low sporulating strain: a strain is considered as low sporulating strain when the spore production is ten times lower than classical strains, for example is between about 104 and 108 per print during 24 hours.

Marker assisted selection: Using linked genetic markers including molecular markers to track trait-determining loci of interest among offspring and through pedigrees.

MAT: 2 unlinked mating loci, mat A and mat B, which determines sexual compatibility and the dikaryotic state.

Mating: The sexual union of two cultures via anastomosis and plasmogamy; methods of obtaining controlled matings between mushroom cultures are well known in the art.

Monokaryon: A haploid culture with a single type (or somatic lineage) of haploid nucleus (cytogenetically represented as N), and which is ordinarily reproductively incompetent, and which does not exhibit typical self/non-self incompatibility reactions with dikaryons, and which may function as a gamete in sexually complementary anastomoses; a ‘line’ which, as with an inbred plant line, transmits a uniform genotype to offspring; a predominantly homoallelic line that mates well and fruits poorly is a putative monokaryon for strain development purposes; see discussion below.

Monokaryotic: Having the character of a monokaryon; haploid.

Mycelium: The vegetative body or thallus of the mushroom organism, comprised of threadlike hyphae.

Mushroom: The reproductive structure of a Pleurotus fungus; a Pleurotus; a cultivated food product of the same name.

Neohaplont: A haploid culture or line obtained by physically dedikaryotizing (reducing to haploid components) a dikaryon; a somatically obtained monokaryon; a derived monokaryon.

Offspring: Descendants, for example of a parent dikaryon, within a single generation; most often used to describe cultures obtained from spores from a mushroom of a strain.

Outbreeding: Mating among unrelated or distantly related individuals.

Parent: An immediate progenitor of an individual; a parent strain is a dikaryon; a parent line is a monokaryon; a dikaryon may be the parent of an F1 dikaryon via an intermediate parent line/monokaryon offspring.

Pedigree-assisted breeding: The use of genealogical information to identify desirable combinations of lines in controlled mating programs.

Phenotype: Observable characteristics of a strain or line as expressed and manifested in an environment.

Plasmogamy: Establishment, via anastomosis, of cytoplasmic continuity leading to the formation of a sexual dikaryon.

Poorly sporulating strain: See Low sporulating strain.

Progenitor: Ancestor, including parent (i.e., the direct progenitor).

Progeny: See Offspring.

Selfing: Mating among sibling lines; see also intramixis.

Sexual compatibility: A condition among different lines having allelic non-identity at the at two unliked mat A and mat B loci, such that two lines are able to mate to produce a stable and reproductively competent dikaryon. The opposite condition, sexual incompatibility, occurs when two lines each have the same alleles at the mat A and mat B locus.

Somatic: ‘Of the vegetative mycelium’.

Spawn: A mushroom culture, typically a pure culture of a dikaryon, typically on a sterile substrate which is friable and dispersible particulate matter, in some instances cereal grain; commercial inoculum for compost; reference to spawn includes reference to the culture on a substrate.

Spore: Part of the mushroom, the reproductive propagule.

Sporeless strain: A mushroom strain is considered as “sporeless” when no visible spore can be observed on spore prints after 24 hours. In fact, the number of residual spores in these strains is so low that they cannot be seen in these conditions. Yet, few spores can be produced by these mushrooms, e.g., between 20-50 spores per spore print during 24 hours. These spores can be detected for example by letting them grow on an appropriate medium and isolated by microscopy.

Spore print: deposit of matured spores from mushroom gills to an appropriate surface such as paper, glass etc.

Stem: Stipe; part of the mushroom, the cap-supporting structure.

Sterile Growth Media: Nutrient media, sterilized by autoclaving or other methods, that support the growth of the organism; examples include agar-based solid nutrient media such as Potato Dextrose Agar (PDA), nutrient broth, and many other materials.

Stipe: see ‘stem’.

Strain: A dikaryon with defined characteristics or a specific identity or ancestry.

Targeted mutagenesis: a process of altering the DNA sequence of at least one specific gene locus.

Tissue culture: A de-differentiated vegetative mycelium obtained from propagation of a differentiated tissue of the mushroom.

Trait conversion: A method for the selective introduction of the genetic determinants of one (i.e., a single-locus conversion) or more desirable traits into the genetic background of an initial strain while retaining most of the genetic background of the initial strain. See ‘Introgressive trait conversion’ and ‘Transformation’.

Transformation: A process by which the genetic material carried by an individual cell is altered by the incorporation of foreign (exogenous) DNA into its genome or cytoplasm; a method of obtaining a trait conversion including a single-locus conversion, or a novel trait.

Yield: The net fresh weight of the harvest crop, normally expressed in kilograms per 100 kilograms of substrate (expressed in percentage).

Yield pattern: The distribution of yield within each flush and among all flushes; influences size, quality, picking costs, and relative disease pressure on the crop and product.

With respect to the definition of monokaryon above, it is noted that monokaryons and homoallelic lines are subject to technical and practical considerations: A monokaryon in classical terms is a haploid culture which is axiomatically entirely homoallelic. In practical terms, for fungal strain development purposes, the definition is broadened somewhat to accommodate both technical limitations and cytological variation, by treating all predominately homoallelic lines as monokaryons. Technical limitations include the fact that genomes contain duplicated DNA regions including repeated elements such as transposons, and may also include large duplications of chromosomal segments due to historical translocation events. Two Pleurotus ostreatus genome reference have been sequenced.

Pleurotus is a heterothallic homobasidiomycete. The mating is controlled by bifactorial tetrapolar genetic system.

When the relationship is one of back-bred descent from an F1 dikaryon, via mating an offspring monokaryon to a parental monokaryon, the representation and commonality of origin of the parental monokaryon genotype in the back-bred dikaryon will both be roughly 75% on average. Somatic selection cultures and tissue selection cultures will effectively have 100% genetic identity with the initial culture, possibly with epigenetic alterations, or rearrangements, or rare mutations, often present at the same rate as in unselected clonal subcultures, and which are virtually impossible to detect. Mutagenized cultures will similarly have effectively 100% genetic identity with their initial culture, except for one or more random point mutations that are impractical to detect. Transformed cultures will typically have at least 99.99% to 100% genetic identity with their initial culture, plus one small piece of exogenous DNA which may or may not be integrated into the Pleurotus genome.

### FIGURE LEGENDS

FIG. 1 shows a picture of the mushroom of the invention, as compared with a Spoppo mushroom.

FIG. 2 shows how to measure the cap diameter and mushroom height.

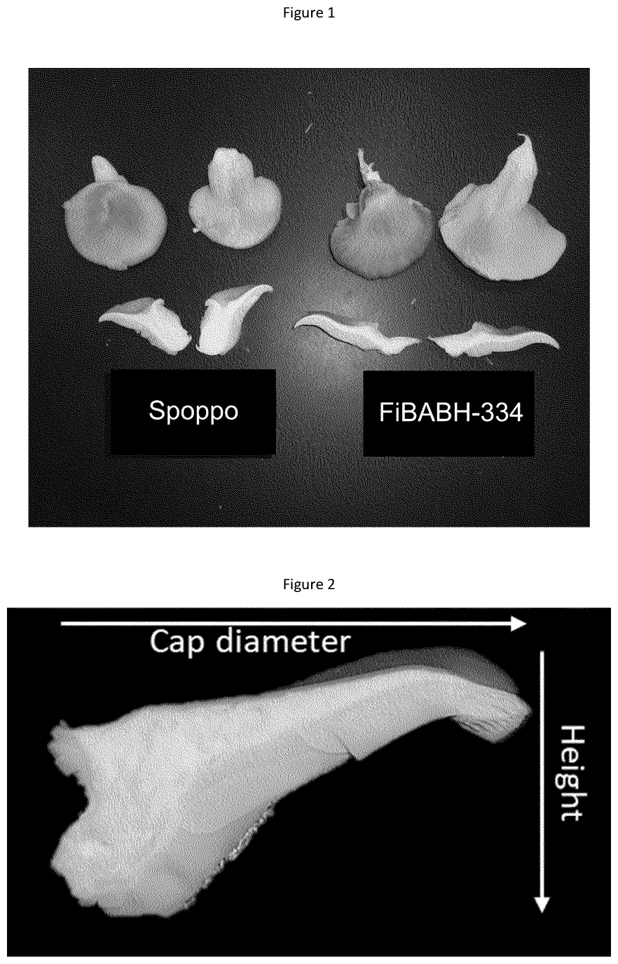

### EXAMPLES

### 1. Yield of the FiBABH-334 Strain

During these trials, mushrooms were grown on fermented wheat straw; the substrate was inoculated with spawn at 40 liters/ton (=2%), packed in 12 kg blocks perforated with 24 holes (20 mm diameter). The trials were performed in greenhouse. The incubation period lasted 18-20 days at substrate temperature of 26-30° C. When the pinset began, the temperature in the air was regulated at 17-18° C., RH of 85-90% with introduction of fresh air and light 12 h/24 h. The strains were tested with 6 replicates, distributed across 3 growing levels. To collect yield, mushrooms were picked daily, the stem was cut then weighted. Data were collected over 2 flushes. The mushroom crop yield of strain FiBABH-334 was found to be equivalent to SPOPPO in 1^{st} and 2^{nd} flush. Cumulative yield difference was found to be not significantly different. as shown in Table 1. Nevertheless, Spoppo reached peak of harvest faster than FiBABH-334 on 1^{st} flush, but the peak of harvest was found similar for both strains on 2^{nd} flush (Table 3).

The yield of two other strains from the state of art, Allerpo and Somycel 3200, were assessed. Concerning the yield, none of the strains outperformed Spoppo. It is either equivalent (Allerpo) or below Spoppo (Somycel 3200). Based on these results, it can be concluded that Allerpo and Somycel 3200 do not have a better yield than FiBABH-334.

TABLE 1
Flush yield of FiBABH-334 and Spoppo after 1 and 2 flushes expressed
kg of mushroom/kg wet substrate (w/w). Standard cultivation and
harvest procedures were used. General t-test analysis: the difference
with FiBABH-334 is significant at p-value ≤ 0.05.

| | Yield | | Yield | | | |
|---|---|---|---|---|---|---|
| | 1^{st} | | 2^{nd} | | Total | |
| | Flush | sd | Flush | sd | yield | sd |
| FiBABH-334 | 16.48% | 1.24% | 6.78% | 3.55% | 23.26% | 2.45% |
| SPOPPO | 15.96% | 2.95% | 6.89% | 2.89% | 22.85% | 5.7% |
| pvalue | 0.698 | | 0.954 | | 0.874 | |

TABLE 2
Flush yield Spoppo and 3200 (A) and Spoppo and Allerpo (B)
after 2 flushes expressed kg of mushroom/kg wet substrate (w/w).
Standard cultivation and harvest procedures were used. General t-test
analysis: the difference with Spoppos significant at p-value ≤0.05.

| | yield | sd | | yield | sd |
|---|---|---|---|---|---|
| SPOPPO | 19.5% | 4.5% | SPOPPO | 17.2% | 4.5% |
| 3200 | 13.9% | 1.6% | ALLERPO | 18.4% | 1.6% |
| p-value | | 0.027 | p-value | | 0.637 |

| | |
|---|---|
| A | B |

TABLE 3
Peak of harvest for FiBABH-334 and Spoppo strains. Results are
expressed in number of days after spawning day. General t-test
analysis: the difference with Spoppo is significative with p-value ≤0.05.

| | 1st Flush | | 2nd flush | |
|---|---|---|---|---|
| | Peak of harvest (days) | sd | Peak of harvest (days) | sd |
| FiBABH-334 | 27.667 | 1.0 | 55.5 | 2.4 |
| SPOPPO | 25.500 | 1.6 | 54.33 | 2.2 |
| p-value | 0.021 | | 0.408 | |

### 2. Color of the Cap of FiBABH-334

The mushroom color was measured using a Minolta Chroma Meter CR-200 (mfd. Japan). Sample sizes of thirty medium sized mushrooms were harvested from the tests and measured to obtain values for the L*a*b parameters. The Chroma Meter readings were randomly taken on the mushroom caps. In the L*a*b system, “L” is a brightness variable with 0 representing complete darkness and 100 representing complete whiteness and “b” value represents blueness (−300)/yellowness (+299). In other words, the darker a mushroom cap color, the lower the L value, and the more yellow a mushroom cap color, the higher b value.

In this case, L value has shown a darker cap color for FiBABH-334 (Table 4).

TABLE 4
Chromameter value L, a, b of SPOPPO and FiBABH-334
strains. General t-test analysis: the difference
with Spoppo is significative with p-value ≤ 0.05

| Strains | L Value | sd | a Value | sd | b Value | sd |
|---|---|---|---|---|---|---|
| Fi-BABH-334 | 69.3 | 4.1 | 1.9 | 0.8 | 14.1 | 1.8 |
| SPOPPO | 73.0 | 5.6 | 2.6 | 1.0 | 15.0 | 2.2 |
| p-value | <0.001 | | <0.001 | | 0.03 | |

### 3. Features of the Cap of FiBABH-334

The morphology of the mushrooms has been measured using a Mitutoyo electronic calliper on 50 to 60 mushrooms/strain per flush.

For each mushroom the cap diameter and the height of the mushroom was measured as shown on FIG. 2.

As shown in table 5 for an equivalent size of cap diameter between Spoppo and FiBABH-334, the strain FiBABH-334 is flatter.

This trait will solve or at least improve and facilitate packaging issue by taking less space, the reduced height is due to the morphology with FiBABH-334 having a short stem. This attribute allows also to reduce the rate of waste (see below).

TABLE 5
Cap diameter and mushroom height values, for FiBABH-334, Spoppo
and Allerpo strains expressed in mm. General t-test analysis:
the difference with SPOPPO is significative with p-value ≤0.05

| | 1st Flush | | 2nd flush | |
|---|---|---|---|---|
| | Cap diameter | Height | Cap diameter | Height |
| FiBABH-334 | 81.3 | 27.5 | 70.2 | 36.4 |
| SPOPPO | 82.6 | 47.4 | 73.5 | 64.5 |
| p-value | 0.57 | <0.0001 | 0.14 | <0.0001 |

| | | | |
|---|---|---|---|
| ALLERPO | 73.1 | 65.2 | n/a |
| p-value | < 0.0001 | <0.0001 | n/a |

### 4. Rate of Waste

Table 6 below shows the percentage of waste for FiBABH-334 and SPOPPO strains (ratio of cut/uncut weight of mushrooms). Bunches are generally cut to separate the mushrooms and to remove the dirty part that has been in contact with substrate. General t-test analysis: the difference with FIBABH-334 is significative with p-value≤0,05.

TABLE 6
1st Flush

| | Percentage of waste | sd | p-value |
|---|---|---|---|
| FiBABH-334 | 11.9% | 1.7% | |
| SPOPPO | 17.6% | 3.5% | 0.005 |
| ALLERPO | 21.4% | 2.5% | 0.001 |

1. A sporeless Pleurotus strain comprising at least one haploid set of chromosomes of the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023.

2. The sporeless Pleurotus strain of claim 1, characterized in that it is selected from the group consisting of:
(a) F1 hybrids produced by mating a monokaryon from FiBABH-334 with a second line;
(b) the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023;
(c) derivatives of the strain (b), said derivatives being cells obtained from said FIBABH-334 strain by somatic selection, tissue culture selection, single spore germination, multiple spore germination, matings between mycelium obtained from monokaryotic or dikaryotic spores from these hybrids, selfing, repeated mating back to the initial culture, mutagenesis, trait conversion, introgressive trait conversion, inbreeding or transformation; and
(d) a monokaryon of the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023.

3. The sporeless Pleurotus strain of claim 2, which is a derivative of the strain as defined in claim 2(b), said derivatives being cells obtained from said FiBABH-334 strain by somatic selection, tissue culture selection, single spore germination, multiple spore germination, matings between mycelium obtained from monokaryotic or dikaryotic spores from these hybrids, selfing, repeated mating back to the initial culture, mutagenesis, trait conversion, introgressive trait conversion, inbreeding or transformation;
comprising at least 99%, or at least 98%, or at least 95%, or at least 92%, or at least 90%, or at least 85%, or at least 80%, or at least 75%, or at least 70%, or at least 65%, or at least 60%, or at least 55%, or at least 50%, or at least 45%, or at least 40%, or at least 30%, or at least 20%, or at least 10%, of the sequence-characterized allelic markers of FiBABH-334 listed in Table A.

4. The sporeless Pleurotus strain of claim 2, which is a derivative of the strain as defined in claim 2(b), said derivatives being cells obtained from said FiBABH-334 strain by somatic selection, tissue culture selection, single spore germination, multiple spore germination, matings between mycelium obtained from monokaryotic or dikaryotic spores from these hybrids, selfing, repeated mating back to the initial culture, mutagenesis, trait conversion, introgressive trait conversion, inbreeding or transformation;
comprising between 80%-100%, or between 60-80%, or between 40-60%, or between 20-40% of the sequence-characterized allelic markers of FIBABH-334 listed in Table A.

5. A sporeless Pleurotus strain of the F2 or F3 generation, descended from the strain FiBABH-334, or from a strain derived from strain FIBABH-334, a representative culture of said line having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number 1-5939; or a sporeless Pleurotus mushroom strain that is derived from the initial strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023.

6. The sporeless Pleurotus strain of claim 5, which is a sporeless Pleurotus strain of the F2 or F3 generation descending from FiBABH-334 or from a strain derived from FiBABH-334, comprising at least about 200 allelic markers out of the 298 sequence-characterized allelic markers of FiBABH-334 listed in Table A.

7. The sporeless Pleurotus strain of claim 5, which is a sporeless Pleurotus strain of the F2 or F3 generation descending from FiBABH-334 or from a strain derived from FiBABH-334, comprising at least 99%, or at least 98%, or at least 95%, or at least 92%, or at least 90%, or at least 85%, or at least 80%, or at least 75%, or at least 70%, or at least 65%, or at least 60%, or at least 55%, or at least 50%, or at least 45%, or at least 40%, or at least 30%, or at least 20%, or at least 10%, of the sequence-characterized allelic markers of FIBABH-334 listed in Table A.

8. The sporeless Pleurotus strain of claim 5, which is a sporeless Pleurotus strain of the F2 or F3 generation descending from FiBABH-334 or from a strain derived from FiBABH-334, comprising between 80%-100%, or between 60-80%, or between 40-60%, or between 20-40% of the sequence-characterized allelic markers of FiBABH-334 listed in Table A.

9. (canceled)

10. The sporeless Pleurotus mushroom strain of claim 5, which is a sporeless Pleurotus mushroom strain that is derived from the initial strain FiBABH-334, and comprises at least 85% of the sequence-characterized allelic markers of FiBABH-334 listed in Table A.

11. A culture of any of the mushroom strain of claim 1, characterized in that:
(a) the total yield performance of the crops of said culture is at least equal to the yield performance of crops of the Spoppo strain of Pleurotus, and
(b) the average total height of the resulting mushrooms, including their stipe and cap, is significantly inferior to the average height of Spoppo mushrooms, and
(c) the average height of the stipes of the resulting mushrooms, is significantly inferior to the average height of Spoppo mushroom stipes, and
(d) the cap of the resulting mushrooms is flatter than the caps of Spoppo mushrooms.

12. A mushroom produced by growing a crop of mushrooms from the strain of claim 1 any of claim 1.

13. Cells, spores, hyphae, or mycelium obtained from the strain of claim 1.

14. (canceled)

15. A product incorporating the strain of claim 1, including spawn, inoculum, processed foods, colonised substrates including grain, substrate and friable particulate matter.

16. A method for producing Pleurotus mushrooms, said method comprising:
a) Inoculating a mushroom growth medium with a sporeless Pleurotus strain of claim 1,
b) Maintaining said inoculated growth medium under conditions conductive to mushroom fruiting and
c) Collecting mushrooms from said growth medium.

17. A method for developing a new sporeless Pleurotus culture, said method comprising applying at least one mushroom strain development technique to the strain FiBABH-334, a representative culture of which having been deposited at the Collection Nationale de Cultures de Microorganismes (CNCM), Institut Pasteur, 25 rue du Docteur Roux, 75724 PARIS Cedex 15, on Mar. 21, 2023, under the CNCM Accession Number I-5939, or to a progeny thereof, to provide a new culture.

18. The method of claim 17, comprising mating a monokaryon of the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023; or mating a sporeless Pleurotus strain comprising at least one haploid set of chromosomes of the strain FiBABH-334, a representative culture of said strain having been deposited under the CNCM Accession Number 1-5939 at the Collection Nationale de Cultures de Microorganismes (CNCM), Institut Pasteur, 25 rue du Docteur Roux, 75724 PARIS Cedex 15, on Mar. 21, 2023, with a second Pleurotus monokaryon or dikaryon.

19. A culture of the mushroom strain of claim 5, characterized in that:
(a) the total yield performance of the crops of said culture is at least equal to the yield performance of crops of the Spoppo strain of Pleurotus, and
(b) the average total height of the resulting mushrooms, including their stipe and cap, is significantly inferior to the average height of Spoppo mushrooms, and
(c) the average height of the stipes of the resulting mushrooms, is significantly inferior to the average height of Spoppo mushroom stipes, and
(d) the cap of the resulting mushrooms is flatter than the caps of Spoppo mushrooms.

20. A mushroom produced by growing a crop of mushrooms from the strain of claim 5.

21. A mushroom produced by growing a crop of mushrooms from the culture of claim 11.

22. Cells, spores, hyphae, or mycelium obtained from the strain of claim 5.

23. Cells, spores, hyphae, or mycelium obtained from the culture of claim 11.

24. A product incorporating the strain of claim 5, including spawn, inoculum, processed foods, colonised substrates including grain, substrate and friable particulate matter.

25. A product incorporating the culture of claim 11, including spawn, inoculum, processed foods, colonised substrates including grain, substrate and friable particulate matter.
